(19) United States
(12) Patent Application Publication
(10) Pub. No.: US 2009/0191242 A1
(43) Pub. Date: Jul. 30, 2009

(54) NOVEL HOMEOPATHIC STRAIN COMPRISING A NOSODE FROM A PROTOZOON OF THE GENUS TRYPANOSOMA

(76) Inventors: Isabelle DELBOSC, Saint Chinian (FR); Bruno ENJALBERT, Saint Chinian (FR)

Correspondence Address: BROWDY AND NEIMARK, P.L.L.C.;624 NINTH STREET, NW, SUITE 300, WASHINGTON, DC, 20001-5303, US

(21) Appl. No.: 12/330,049

(22) Filed: Dec. 8, 2008

(30) Foreign Application Priority Data
Dec. 7, 2007 (FR) 07 08533

(51) Int. Cl. A61K 39/005; A61K 35/68; A61P 33/02

(52) U.S. Cl. 424/269.1; 424/780

(57) ABSTRACT

The present invention relates to a novel homeopathic strain characterized in that it comprises a nosode from a protozoon of the genus Trypanosoma.

The invention also relates to the use of this homeopathic strain in the preparation of a medicament intended for the treatment of the clinical pathological signs similar to those observed in the trypanosomiases.

It also relates to a process for the preparation of this homeopathic strain as well as a process for the preparation of a homeopathic medicinal speciality comprising said homeopathic strain.

The present invention relates to a novel homeopathic strain characterized in that it comprises a nosode from a protozoon of the genus Trypanosoma.

The invention also relates to the use of this homeopathic strain in the preparation of a medicament intended for the treatment of symptoms similar to those found in the trypanosomiases.

By similar to those found in the trypanosomiases is meant according to the invention the symptoms recognized as being induced during trypanosomiases, whether or not they are induced by a trypanosomiasis.

It also relates to a process for the preparation of this homeopathic strain.

Trypanosoma is a genus of the order of the Trypanosomatida, a group of parasitic protists. Some twenty species exist which infect various vertebrates, including humans, causing diseases, the trypanosomiases. The human trypanosomiases are endemic in two regions of the world: Africa and South America. There are also, including in other regions of the world, trypanosomiases which affect other species but which are not dangerous to humans; they can however cause significant damage to farm animals.

Most of the species are transmitted by hematophagous invertebrates such as insects or leeches.

The trypanosomes responsible are Trypanosoma brucei gambiense, Trypanosoma brucei rhodesiense, Trypanosoma cruzi, Trypanosoma evansi, and still more particularly Trypanosoma brucei gambiense, Trypanosoma brucei rhodesiense. Their life cycle requires two hosts for development and reproduction. In addition to humans, one of its main carriers is the tsetse fly (Glossina palpalis). In humans the trypanosoma develops in the blood, the bone marrow and the cephalo-rachidian liquid. It is also found in antelopes and bovids. Its average size is 20 microns long by 2 to 3 microns wide, and it is entirely mobile.

In its propagation cycle, when the tsetse fly bites an infected person, the trypanosome is ingested and multiplies in the fly's intestine. The asexual multiplication cycle takes place in the salivary glands. The development cycle of the trypanosome in the fly lasts between 18 and 25 days at the end of which it can be transmitted to humans by biting.

In humans the period of invasion of the parasite occurs after incubation for 10 to 15 days. Propagation also occurs via the lymph and leads to the appearance of fevers, as well as hypertrophy of the liver (hepatomegaly) and/or of the spleen (splenomegaly). This is followed by invasion of the meninges and of the cephalo-rachidian liquid causing headaches then motor disturbances as well as reflex disorders and finally psychological disorders. The disease is fatal within a few months.

The art of prescribing a homeopathic medicament is subject to the similarity law. When an individual exhibits a functional or physical clinical sign, the rule is to prescribe a homeopathic strain indicated for combating a similar clinical sign. The indications of a homeopathic strain are established by the pathogenesis of this strain. Strain, pathogenesis and indications define the materia medica of the homeopathic medicament.

Thus the clinical symptoms such as fever, chills, headache, particularly of persistent headaches, adenopathy, arthralgia, anaemia, recurrent circinate erythema, spontaneously healing inoculation chancre, lymphadenitis, sub-cutaneous cellulitis with slightly inflammatory oedema, sensitive adenopathies with progressive induration, myocarditis, hypertrophic cardiomyopathy, mega-oesophagus, megacolon, deglutition disorder, hepatosplenomegaly, difficulties with concentration, cognitive disorders, apraxia, confusion, mnesic disorders, time-space disorientation, ataxia, day-night inversion, insomnia, mental deterioration, peripheral neuropathy, vertigo, tinnitus, tremor, incontinence, motor deficit, depressive syndrome, psychotic syndrome, emaciation, anorexia, oedemas of the extremities, nephrotic syndrome, clinical symptoms present in the trypanosomiases, whether or not they are induced by a trypanosomiasis, should be able to be treated with a medicament the homeopathic materia medica of which comprises Trypanosoma, particularly a nosode from a protozoon of the genus Trypanosoma.

One of the objectives of the present invention is to provide a novel medicament in order to attenuate, or even treat, the clinical signs similar to those found in the trypanosomiases, with fewer or even non-existent side effects compared with existing medicaments.

The Applicant has surprisingly discovered that nosodes from trypanosomes can be used in the care or prevention of similar symptoms identical to those found in the trypanosomiases, whilst exhibiting few or even no side effects.

Thus a subject of the invention is a homeopathic strain which comprises a nosode from a protozoon of the genus Trypanosoma,

A nosode is a particular type of homeopathic medicament taken from the causal agent of a disease in order to treat similar functional or physical disorders found in other diseases. By definition, the nosode has no vaccinal role, since it does not confer immunity. However in certain circumstances, for health reasons, a person skilled in the art is obliged to reconcile himself, although the medicament is then less effective, to use as a homeopathic strain a pathogenic germ vaccine such as for example in the case of Influenzinum.

In the case of the present invention, it is preferred to use a nosode understood in the sense of its generally accepted definition. However, as the invention can also be implemented using a vaccine, a toxin, an anatoxin, as well as any infectious or non-infectious antigen element, the definition of the term nosode is extended to vaccines, toxins and anatoxins, as well as to any infectious or non-infectious antigen element, originating from a protozoon of the genus Trypanosoma.

Thus according to the invention, a nosode can be
a contaminated or contaminating product originating from an individual carrying a pathology and/or its causal agent, such as for example a tissue and/or all or part of an organ from said individual, such as for example the liver, heart, kidney, thyroid or lung,
a secretion or excretion from said individual, such as for example faeces, urine, pus, blood, saliva, the cephalo-rachidian liquid, an exudate, a serosity,
or also a portion of necrotic tissue or of a tumour or any cell from said individual, having been or being in contact with said causal agent of said pathology.

These pathological products can be taken from individuals contaminated with Trypanosoma, who may or may not be treated.

A nosode can also be a biological derivative such as a vaccine, a toxin, an anatoxin, as well as any infectious or non-infectious antigenic element.

It can also be produced from the causal agent of the pathology, for example from microorganisms, for example by culture in vivo or in vitro.

It is also understood that it is possible to use a mixture of different nosodes in order to realise the invention.

The nosodes can be used in the crude state, for example for preparing a tincture, or also in the form of a derivative, such as for example an autolysate.

The nosodes can be of animal or human origin.

According to the invention, the nosode introduced into the novel homeopathic strain originates from an individual contaminated with a protozoon of the genus Trypanosoma.

Preferably according to the invention, the nosode comes from tissues (liver, heart, kidney, brain etc.) contaminated with a protozoon of the genus Trypanosoma, whether from an artificial, accidental or natural host, preferably originating from a laboratory rat or secondarily from any vector of the whole parasite used.

By homeopathic strain according to the invention is meant a therapeutic preparation comprising at least one nosode originating from an individual contaminated with a protozoon of the genus Trypanosoma.

It is also understood that according to the invention it is possible to use a mixture of different nosodes for preparing the novel homeopathic strain.

According to the invention the homeopathic strain can comprise 100% nosode.

According to the invention the nosodes used for preparing the homeopathic strain according to the practice conventionally used in the field of homeopathy, can be treated in order to remove any health risk, then diluted and dynamized.

According to the invention, the nosode can take any form compatible with the preparation of a homeopathic strain. For example and without limitation, the nosode can be dried.

According to the invention, the nosode can be used in the crude state or suitably ground.

According to the invention the nosode used can be constituted by a product contaminated as defined previously, whatever the stage of development of the protozoon of the genus Trypanosoma, (trypomastigote, epimastigote, choanomastigote, amastigote, opsithomastigote, promastigote, metacyclic or procyclic), etc.

According to the invention any protozoon of the genus Trypanosoma can be used for the preparation of the homeopathic strain.

Thus, the protozoon of the genus Trypanosoma can be chosen from Trypanosoma blanchardi, Trypanosoma bourrouli, Trypanosoma boylei, Trypanosoma brazali, Trypanosoma brucei (Trypanosoma b. brucei), Trypanosoma castellanii, Trypanosoma cazalboui, Trypanosoma celeslinoi, Trypanosoma congolense, Trypanosoma conorhini, Trypanosoma colli, Trypanosoma cruzi, Trypanosoma cuniculi, Trypanosoma dimorphen, Trypanosoma duttoni, Trypanosoma equiperdum, Trypanosoma equinum, Trypanosoma escomeli, Trypanosoma evansi, Trypanosoma fordi, Trypanosoma gambiense (Trypanosoma b. gambiense), Trypanosoma granulosum, Trypanosoma grayi, Trypanosoma guernei, Trypanosoma hominis, Trypanosoma inopinatum, Trypanosoma lanfranchii, Trypanosoma leptodactyli, Trypanosoma lewisi, Trypanosoma melophagium, Trypanosoma musculi, Trypanosoma nabiasi, Trypanosoma nanam, Trypanosoma nepveui, Trypanosoma nigeriense, Trypanosoma ocellati, Trypanosoma ovium, Trypanosoma pecaudi, Trypanosoma percae, Trypanosoma primatum, Trypanosoma rabinowitschi, Trypanosoma rangeli, Trypanosoma* ratarium, Trypanosoma rhodesiense (Trypanosoma b. rhodesiense), Trypanosoma scardinil, Trypanosoma scylli, Trypanosoma simiae, Trypanosoma suis, Trypanosoma theileri, Trypanosoma ugandae, Trypanosoma ugandense, Trypanosoma uniforme, Trypanosoma varani, Trypanosoma vickersae, Trypanosoma vivax, ablepharoplastic Trypanosoma, Trypanosoma of the eel, Trypanosoma americaine.

Among the protozoa described above, the following: Trypanosoma cruzi, Trypanosoma evansi are more particularly adopted, and still more particularly Trypanosoma brucei gambiense, Trypanosoma brucei rhodesiense.

The useful properties of the homeopathic strain comprising at least one nosode as defined in the present invention justify their use for the preparation of a medicament, particularly of a medicament intended for the treatment or prevention of the clinical symptoms similar to those found in the trypanosomiases such as fever, chills, headache, particularly persistent headaches, adenopathy, arthralgia, anaemia, recurrent circinate erythema, spontaneously healing inoculation chancre, lymphadenitis, sub-cutaneous cellulitis with slightly inflammatory oedema, sensitive adenopathies with progressive induration, myocarditis, hypertrophic cardiomyopathy, mega-oesophagus, megacolon, deglutition disorder, hepatosplenomegaly, difficulties with concentration, cognitive disorders, apraxia, confusion, mnesic disorders, time-space disorientation, ataxia, day-night inversion, insomnia, mental deterioration, peripheral neuropathy, vertigo, tinnitus, tremor, incontinence, motor deficit, depressive syndrome, psychotic syndrome, emaciation, anorexia, oedemas of the extremities, nephrotic syndrome.

Thus a subject of the invention is also a medicament which comprises at least the homeopathic strain of the invention.

A particular subject of the present invention is also the use of the homeopathic strain as described previously in the preparation of a medicament intended for the treatment or prevention of the functional or physical clinical symptoms similar to those found in trypanosomiasis.

A subject of the invention is also the use of the homeopathic strain as described previously, in the preparation of a medicament intended for the treatment or prevention of fever, chills, headache, particularly of persistent headaches, adenopathy, arthralgia, anaemia, recurrent circinate erythema, spontaneously healing inoculation chancre, lymphadenitis, sub-cutaneous cellulitis with slightly inflammatory oedema, sensitive adenopathies with progressive induration, myocarditis, hypertrophic cardiomyopathy, mega-oesophagus, megacolon, deglutition disorder, hepatosplenomegaly, difficulties with concentration, cognitive disorders, apraxia, confusion, mnesic disorders, time-space disorientation, ataxia, day-night inversion, insomnia, mental deterioration, peripheral neuropathy, vertigo, tinnitus, tremor, incontinence, motor deficit, depressive syndrome, psychotic syndrome, emaciation, anorexia, oedemas of the extremities, nephrotic syndrome, particularly in the diseases the clinical signs of which are similar to those found in the trypanosomiases.

The homeopathic strain according to the invention can be used particularly as a medicament in mammals, more particularly in humans. It causes in patients exhibiting clinical signs identical to those found in the trypanosomiases an improvement in their state, and even a cure.

The homeopathic strains according to the invention can moreover comprise at least one other therapeutically active ingredient, whether active on the same symptom or on a different symptom of the same pathology or different pathology for simultaneous or separate use or use spread over time, in particular during treatment in a subject suffering from the abovementioned pathological signs.

It may of course be appropriate to add adjuvants which are usual in human medicine, for example in order to enhance the activity of the medicament.

The medicament according to the invention can be combined with one or more other homeopathic medicaments, and/or with one or more other conventional medicaments which are active against the clinical pathological signs similar to those found in the trypanosomiases.

According to the invention, the nosode can be used in the homeopathic strain, in a mixture with one or more inert, i.e. pharmaceutically inactive and non-toxic, excipients or vehicles. For example, there can be mentioned saline, physiological, isotonic, buffered solutions, etc., compatible with a pharmaceutical use and known to a person skilled in the art.

The homeopathic strain can contain one or more agents or vehicles chosen from the dispersing agents, solubilizers, stabilizers, preservatives, etc. Agents or vehicles which can be used in (liquid and/or injectable and/or solid) formulations are in particular methylcellulose, hydroxymethylcellulose, carboxymethylcellulose, cyclodextrins, polysorbate 80, mannitol, gelatin, lactose, vegetable or animal oils, acacia, etc.

Preferably, vegetable oils are used.

The strain can be formulated in the form of granules, globules, pearls, pills, syrup, a solution, an injectable suspension, a gel, oil, tablet, suppository, powder, gelatin capsule, capsule, ointment, cream, lotion, etc., optionally by means of pharmaceutical forms or devices ensuring a controlled and/or delayed release. For this type of formulation, an agent such as saccharose, lactose, cellulose, carbonates or starches are advantageously used.

Preferably, the preferred formulation of the invention can be a dose tube of globules.

According to the invention the medicaments can be formulated for the digestive rather than the parenteral route. Preferably the medicaments can be formulated for the sublingual route.

According to the invention the nosode is advantageously present in the medicament in effective homeopathic doses, (of 1 HD to 30 HC, or even 1,000,000 K)

According to the invention, the administration can be carried out by any method known to a person skilled in the art, such as for example a dose of globules (or granules) weekly or bimonthly or even monthly for preventive treatment, or even daily or several times daily if the clinical signs are more pronounced. The administration also depends on the response of the patient to the treatment.

In general the dose according to the invention used in the course of the treatment is the minimum dose for obtaining the desired therapeutic effect and conventionally used in the case of homeopathic treatment of the clinical pathological signs identical to those found in the trypanosomiases.

If necessary, the daily, weekly, monthly or quarterly dose can be given in two, three, four, five, six or more administrations, or by multiple sub-doses administered at appropriate intervals during the period determined by the objectives of treating symptoms or prevention.

A person skilled in the art knows how to adapt the quantities of nosode used depending on multiple factors, in particular the administration route, duration of administration, time of administration, the patient's response, symptom to be treated, the preventive function to be targeted, the speed of elimination of the compound, the different product or products used in combination with the compound, the age, weight and physical condition of the patient, as well as his medical history, and any other information known in medecine.

The preparation of the medicament of the invention is carried out with recognized methods currently used in the homeopathic pharmacopoeia. As all tissues, organs and organisms harbouring Trypanosoma can be used for preparing the medicament, the invention extends to the other homeopathic strains which have been in contact with all the protozoa of the genus Trypanosoma.

Depending on pharmacological requirements of each country, the guarantee of the harmlessness of the medicament of the invention can be ensured by the production of the “mother tincture”.

Any process for the preparation of a mother tincture known in the field of pharmacy can be used according to the invention.

A subject of the invention is also a process for the preparation of a homeopathic strain, characterized in that
in a first stage at least one nosode previously taken from its initial strain can be macerated in dilute ethyl alcohol in order to produce a mother tincture, for a period comprised between 1 and 50 days, preferably between 10 and 20 days, at a temperature comprised between 5 and 25°C., preferably between 10 and 20° C.;
in a second stage, the maceration obtained can be pressed in order to obtain a fraction of useable mother tincture;
in a third stage, the mother tincture obtained in the preceding stage is diluted and dynamized (vigorous stirring before each dilution) according to the Hahnemannian or Korsakovian methods well known to a person skilled in the art, at dilutions comprised between 1 HD and 30 HC or 1 K and 10,000,000 K, preferably between 9 HC and 30 HC. (for the so-called Korsakovian dilutions in a single flask, the dilutions are comprised between 10 and 10,000 K).

Of course, according to the invention and depending on the momentary requirements, the process according to the invention can moreover comprise one or more of the following stages (without this being limitative):
freezing of the nosode before production of the mother tincture;
dehydration over a long period in an oven before production of the mother tincture;
finer grinding before production of the mother tincture;
freezing of the mother tincture after production.

According to the invention the ratio of the alcohol to the nosode (vol/vol) cannot be less than 10 (1 DH) and can be up to 1,000,000 (3 CH). The ideal ratio is 1% (which corresponds to the first centesimal Hanemannian dilution: 1 CH).

For example the mother tincture can be produced with a ratio of 1% (1CH) for contaminated tissues or 1/10,000 (2 CH) for contagious vector insects.

According to the invention the ethyl alcohol used can be diluted with distilled water. The degrees of pure alcohol which can be used according to the invention, can be 60, 70, 80, or 90.

A subject of the invention is finally a process for the preparation of a homeopathic medicinal speciality, characterized in that
in a first stage infinitesimal Hahnemannian dilutions are carried out until the desired concentration is obtained
in a second stage, the support chosen is impregnated with the solution obtained in the first stage.

According to the invention the desired concentrations are concentrations which can be comprised between 1 HD and 30 CH or 1K and 10,000,000K, preferably between 9 CH and 30 CH.

Also according to the invention, the support can be chosen from granules, globules, pearls and pills.

The following examples illustrate the invention without however limiting it.

### EXAMPLE 1

### The Following Tissues are Taken From a Wistar Rat (Charles River/Iffa-Credo Breeding Centre) Contaminated With Trypanosoma Rhodesiense and Free of Any Other Pathology: Liver, Kidneys, Heart, lungs and Brain.

This nosode is ground in a mortar.

It is immersed in 70° ethyl alcohol diluted with distilled water.

The ratio of the alcohol to the nosode is 100 (1 CH). (1 volume of nosode per 99 volumes of alcohol)

The alcoholic tincture obtained is macerated cold at +15° for ten days.

Subsequently the alcoholic tincture is pressed in order to obtain a fraction of “useable mother tincture”.

The dilution and the dynamization of this alcoholic tincture are carried out according to the Hahnemannian methods.

It is carried out in a laminar flow cabinet which filters the air until fewer than 100 particles are obtained per 30 litres of air (“clean room” standards).

The solvent is totally purified 70° ethyl alcohol.

The dilution flasks are washed with distilled water, then heated at 200° c. for 75 minutes and filled with purified air similar to that of the clean room before being used for the dilutions.

Hahnemannian infinitesimal dilutions are carried out up to 15 CH.

The pharmaceutical supports used are sugar-coated lactose and saccharose globules.

These globules are impregnated with the infinitesimal dilution of 15 CH.

The globules are packaged in 1 gram dose tubes.

The entire tube is intended to be administered, in a single weekly dose.

### EXAMPLE 2

### Glossina Palpalis Flies Contaminated with Trypanosoma Rhodesiense and Free of Any Other Pathology, are Immersed Alive in 60° Ethyl Alcohol Diluted With Distilled Water

The ratio of the alcohol to the nosode is 10,000 (2 CH). (1 volume per 9,999 volumes of alcohol)

This ratio is determined by weighing and adjusted by addition of 60° alcohol after the death of the glossinae.

The alcoholic tincture obtained is macerated cold at +15° for 20 days.

Subsequently the alcoholic tincture is pressed in order to obtain a fraction of “useable mother tincture”.

The dilution and the dynamization of this alcoholic tincture is carried out according to the Hahnemannian methods.

It is carried out in a laminar flow cabinet which filters the air until less than 100 particles per 30 litres of air are obtained (“clean room” standards). The solvent is totally purified 70° ethyl alcohol.

The dilution flasks are washed with distilled water, then heated at 200° C. for 75 minutes and filled with purified air similar to that of the clean room before being used for the dilutions.

Hahnemannian infinitesimal dilutions are carried out up to 15 CH.

The pharmaceutical supports used are sugar-coated lactose and saccharose globules.

These globules are impregnated with the infinitesimal dilution of 15 CH.

1. Homeopathic strain characterized in that it comprises a nosode from a protozoon of the genus Trypanosoma.

2. Homeopathic strain according to claim 1, characterized in that the nosode is constituted
a. by a contaminated or contaminating product originating from an individual carrier of a pathology and/or of its causal agent, such as a tissue and/or all or part of an organ from said individual,
b. a secretion or an excretion from said individual,
c. or also a portion of necrotic tissue or of a tumour or any cell of said individual being or having been in contact with said causal agent of said pathology, and/or
d. a biological derivative such as a vaccine, a toxin, an anatoxin,
e. and/or any infectious or non-infectious antigenic element, and/or
f. any element produced from the microorganism.

3. Homeopathic strain according to claim 1, characterized in that the protozoon of the genus Trypanosoma is chosen from Trypanosoma blanchardi, Trypanosoma bourrouli, Trypanosoma boylei, Trypanosoma brazali, Trypanosoma brucei (Trypanosoma b. brucei), Trypanosoma castel/anii, Trypanosoma cazalboui, Trypanosoma celeslinoi, Trypanosoma congolense, Trypanosoma conorhini, Trypanosoma colli, Trypanosoma cruzi, Trypanosoma cuniculi, Trypanosoma dimorphen, Trypanosoma duttoni, Trypanosoma equiperdum, Trypanosoma equinum, Trypanosoma escomeli, Trypanosoma evansi, Trypanosoma fordi, Trypanosoma gambiense (Trypanosoma b. gambiense), Trypanosoma granulosum, Trypanosoma grayi, Trypanosoma guemei, Trypanosoma hominis, Trypanosoma inopinatum, Trypanosoma lanfranchii, Trypanosoma leptodactyli, Trypanosoma lewisi, Trypanosoma melophagium, Trypanosoma musculi, Trypanosoma nabiasi, Trypanosoma nanam, Trypanosoma nepveui, Trypanosoma nigeriense, Trypanosoma ocel/ati, Trypanosoma ovium, Trypanosoma pecaudi, Trypanosoma percae, Trypanosoma primatum, Trypanosoma rabin0 witschi, Trypanosoma rangeli, Trypanosomaratarium, Trypanosoma rhodesiense (Trypanosoma b. rhodesiense), Trypanosoma scardinil, Trypanosoma scylli, Trypanosoma simiae, Trypanosoma suis, Trypanosoma theileri, Trypanosoma ugandae, Trypanosoma ugandense, Trypanosoma uniforme, Trypanosoma varani, Trypanosoma vickersae, Trypanosoma vivax, ablepharoplastic Trypanosoma, Trypanosoma of the eel, Trypanosoma americaine.

4. Homeopathic strain according to claim 3, characterized in that the protozoon of the genus Trypanosoma is chosen from Trypanosoma brucei gambiense, Trypanosoma brucei rhodesiense, Trypanosoma cruzi, Trypanosoma evansi

5. Homeopathic strain according to claim 1, characterized in that the nosode is composed entirely of the homeopathic strain.

6. Medicament characterized in that it comprises at least the homeopathic strain as described in claim 1.

7. A method for the treatment or prevention of the functional or physical clinical symptoms similar to those found in trypanosomiasis, comprising administering the homeopathic strain as described in claim 1.

8. A method for the treatment or prevention of fever, chills, headache, particularly of persistent headaches, adenopathy, arthralgia, anaemia, recurrent circinate erythema, spontaneously healing inoculation chancre, lymphadenitis, sub-cutaneous cellulitis with slightly inflammatory oedema, sensitive adenopathies with progressive induration, myocarditis, hypertrophic cardiomyopathy, of the mega-resophagus, megacolon, deglutition disorder, hepatosplenomegaly, difficulties with concentration, cognitive disorders, apraxia, confusion, mnesic disorders, time-space disorientation, ataxia, day-night inversion, insomnia, mental deterioration, .peripheral neuropathy, vertigo, tinnitus, tremor, incontinence, motor deficit, depressive syndrome, psychotic syndrome, emaciation, anorexia, oedemas of the extremities, nephrotic syndrome, particularly in the diseases the clinical signs of which are similar to those found in the trypanosomiases, comprising administering the homeopathic strain as described in claim 1.

9. Process for the preparation of the homeopathic strain as described in claim 1, characterized in that
in a first stage at least one nosode previously taken from its initial strain can be macerated in dilute ethyl alcohol in order to produce a mother tincture, for a period comprised between 5 and 50 days, preferably between 10 and 20 days, at a temperature comprised between 5 and 25° C., preferably between 10 and 20° C.;
in a second stage, the maceration obtained can be pressed and/or filtered in order to obtain a fraction of useable mother tincture;
in a third stage, the mother tincture obtained in the preceding stage is diluted and dynamized according to the Hahnemannian or Korsakovian methods, up to dilutions comprised between 1 HD and 30 CH or 1K and 10,000,000K, preferably between 9 CH and 30 CH.

10. Process for the preparation of a homeopathic medicinal speciality, characterized in that
in a first stage Hahnemannian or Korsakovian infinitesimal dilutions of a homeopathic strain are carried out as described in claim 1, until the desired concentration is obtained;
in a second stage, the chosen support is impregnated with the solution obtained in the first stage.
